(19) United States
(12) Patent Application Publication
(10) Pub. No.: US 2014/0197571 A1
(43) Pub. Date: Jul. 17, 2014

(54) PREVENTION OF FRETTING CREVICE CORROSION OF MODULAR TAPER INTERFACES IN ORTHOPEDIC IMPLANTS

(71) Applicant: Jeremy Gilbert, Fayetteville, NY (US)

(72) Inventor: Jeremy Gilbert, Fayetteville, NY (US)

(73) Assignee: Syracuse University, Syracuse, NY (US)

(21) Appl. No.: 14/072,296

(22) Filed: Nov. 5, 2013

Related U.S. Application Data
(60) Provisional application No. 61/722,459, filed on Nov. 5, 2012.

(51) Int. Cl. A61L 27/34; A61L 27/50

(52) U.S. Cl. CPC A61L 27/34; A61L 27/50
USPC 264/263; 428/220; 523/113

(57) ABSTRACT

A thin high strength polymer based self-reinforced composite (SRC) made from a biocompatible and commonly used orthopedic implant material is interposed between the hard connections of the modular taper interface of an orthopedic implant. A thick film of this ultra high strength SRC with the fibers oriented to maximally resist the fretting motion and wear, can be interposed between the modular components to result in a relatively soft, non-conducting interposed layer that will prevent contact between hard interfaces and reduce or eliminate the oxide film abrasion associated with this mechanism. Alternatively, the SRC may be formed using surface control methods resist fretting, such as by using sticky-compliant surfaces and channels for fluid, among others.

### CROSS-REFERENCE TO RELATED APPLICATIONS

The present application claims priority to U.S. Provisional Application No. 61/722,459, filed on Nov. 5, 2012.

### BACKGROUND OF THE INVENTION

1. Field of the Invention

The present invention relates to orthopedic implants and, more specifically, the preventions of fretting corrosion in the modular taper connections of orthopedic implants.

2. Description of the Related Art

Corrosion of total joint replacements like hip prostheses is a serious and significant problem in today's designs. One main location where this serious form of degradation takes place is at the modular taper junctions that are present in virtually all joint replacements, but particularly with respect to hip replacements.

Modularity allows implants to have several significant advantages in terms of materials selection, geometry, ease of insertion, and ease of revision. In modular implants, portions of the implant are made from separate components which can be mixed and matched at the time of surgery to provide the best combination of materials and geometries for a specific patient's joint replacement. These modular designs require the presence of a junction or interface between components that serves as the location where the individual parts of the implant are joined together. These junctions are typically modular taper interfaces, conical (or elliptical) in shape, that consist of a taper of low angle (e.g., 5 degrees 40 minutes, or other shape) that provides a tight, secure junction to hold the two adjacent components in place.

In a total hip replacement, for example, there may be modular taper junctions between the head of the femoral component and its neck, between the neck and the stem, and between the top (proximal) and bottom (distal) portions of the stem. In the acetabular component, there may be taper interfaces between the cup and the acetabular shell in which is it housed. Other modular taper interfaces may also be present. These modular components may be present between adjacent metal parts, or between metal and ceramic parts. In all of these cases, the tapers have characteristics that make them prone to a type of corrosion attack known as fretting crevice corrosion. In this degradation process, the taper interfaces result in a very tight crevice-like junction in which fluid from the body can penetrate and they are subject to very high cyclic mechanical loading due to the activities of daily living (e.g., walking). The metal alloys used in these joint replacements have multiple requirements that include very high strength and fracture resistance and very high corrosion resistance. The alloys used are typically from one of several alloy systems that include titanium alloys, cobalt-chromium alloys, surgical grade stainless steels, zirconium alloys, and tantalum alloys. (The first three representing a preponderance of all total hip replacements).

When these modular junctions are created by bringing components together in the body, the combination of loading, crevice geometry and solution within the crevice can lead to a severe fretting corrosion attack at the modular taper junction. When the metal alloy surfaces are exposed to aqueous solutions and subjected to fretting motion (small scale cyclic rubbing-like motion at the interface in the range of 100 um or less), the surface oxide thin films that naturally occur on the surfaces of these metal alloys and which provide the corrosion resistance of these alloys, are abraded and the underlying metal is rapidly corroded until the passive oxide films can re-establish themselves (a process which occurs within milliseconds). With activities of daily living, fretting abrasion can occur at modular taper interfaces in the range of 3 to 30 um between the two opposing sides and result in repetitive damage of the oxides. The water present within these crevices can then react with the bare, unoxidized metal to reform the oxide film on the surface, but the byproduct of these reactions can include metal ions and metallic-based particles being released into the crevice and the generation of hydrogen ions (i.e., acids) into the crevice and electrons into the metal. Repeated cyclic loading of these interfaces can set up a process that can lead to severe local crevice solution chemistries of very low pH and very high oxidizing conditions and which can release large amounts of corrosion debris and corrosion byproducts that can damage the taper itself and can induce adverse biological reactions.

There are several consequences of this form of corrosion attack that includes loss of mechanical function of the device, and release of ions and particles into the body which can cause several significant adverse biological responses that include osteolysis and pseudotumor formation.

### BRIEF SUMMARY OF THE INVENTION

It is therefore a principal object and advantage of the present invention to prevent the processes of mechanically assisted crevice corrosion in modular taper connections of orthopedic implants.

In accordance with the foregoing objects and advantages, the present invention minimizes or eliminates the corrosion attack of these modular tapers by addressing one or more of the underlying conditions at the taper that causes the mechanisms of fretting crevice corrosion. The present invention seeks to minimize the crevice-like geometry within modular taper interfaces, to minimize or eliminate the fretting motion between taper parts, and to introduce materials and geometries that prevent hard-on-metal contacts that cause fretting corrosion damage by providing materials and a topographic design approach that alleviates or minimizes the conditions that allow fretting crevice corrosion of modular taper interfaces in total joint replacements to occur. The present invention applies to all total hip replacements used today, and may be applicable to other tapered or crevice-like interfaces in medical devices where fretting crevice corrosion (otherwise known as mechanically assisted crevice corrosion) can occur.

The present invention addresses fretting crevice corrosion by addressing crevice solution chemistry changes due to fretting crevice corrosion that adversely impact the tapers. The present invention also addresses asperity-asperity contacts that result in oxide film fracture and repassivation, and which results in highly accelerated corrosion attack. The present invention further addresses material contact across the taper interface that can reduce or eliminate oxide film disruption (and the associated accelerated corrosion attack).

The present invention accomplished these tasks by creating a taper interface topography that can provide load transfer across the interface, distribute the contact stresses throughout the taper and not compromise the fatigue resistance of the taper junction, while allowing extensive fluid exchange between the taper region and the outside solution. In the design approach of the present invention, specific interface geometries are introduced, such as grooves or pillars on one side of the taper junction that provide sufficient contact and load transfer, while also having an open solution region where buildup of hydrogen ions, or other degradation products will not dramatically alter solution chemistry and lead to adverse run-away corrosion attack as is seen in current taper designs.

The present invention also accomplishes these tasks by creating a taper interface material and topography that results in a sticky and compliant contact between sides of the taper junction such that there is no relative motion between sides of the interface (i.e., sticky), but the tapers can deform to accommodate the stresses and strains transmitted across the interface. The present invention also uses high strength polymer fiber-like or film-like geometries that can withstand the loading across the interface, inhibit metal-on-metal (or ceramic on metal) contact, while also providing a sticky-compliant junction with space for fluid exchange. Acceptable candidate fiber and film types for use with the present invention include highly oriented, ultra high molecular weight polyethylene (UHMWPE), fibers of poly ether-ether ketone (PEEK), as well as self-reinforced composite constructs of these fibers. Thus, the present invention addresses fretting crevice corrosion behavior by designing interfacial geometries that maximally resist fretting motion while preserving good adhesion and transfer of load by taking advantage of sticking-compliance relationship (porous, columnar surfaces) and open access of fluid to the crevice geometry. The present invention also addresses surface modification approaches to enhance the surface oxide's resistance to fretting corrosion damage by adjusting the surface characteristics (hardness, inertness) by, for example, interposing appropriate films (e.g., PEEK thin film) to inhibit oxide abrasion.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of surface geometric structures to provide high interfacial adhesion according to the present invention;

FIGS. 2(a) and (b) are graphs of fretting corrosion test results for PEEK fibers placed between stainless steel pin and disk, including a comparison of stainless steel on stainless steel without PEEK fibers for comparison;

FIGS. 3(a) and (b) are graphs of the coefficient of friction and work per cycle for fretting corrosion tests with PEEK fibers interposed, where frictional measurements are consistent between fiber types and work results show the fibers allow continued sliding at high loads (and high contact stresses).

FIG. 4 shows a scanning electron microscopic image of two PEEK fibers after fretting corrosion testing with the stainless steel disk below;

FIG. 5 is a plot of coefficient of friction over cycles for pin on disk testing of the present invention;

FIG. 6 is a graph of tangential load verses displacement for pin on disk testing of the present invention;

FIG. 7 is a graph of nominal current density over time for pin on disk testing of the present invention;

FIG. 8 is a graph of relative normalized intensity verses energy for pin on disk testing of the present invention;

FIG. 9 is a graph of relative normalized absorbance verses wavenumber for pin on disk testing of the present invention;

FIG. 10 is a post-test SEM image of pin on disk testing of the present invention;

FIG. 11 is a post-test SEM image of pin on disk testing of the present invention;

FIG. 12 is a post-test FTIR spectra of pin on disk testing of the present invention;

FIG. 13 is a post-test FTIR spectra of pin on disk testing of the present invention;

FIG. 14 is a post-test optical microscope image of pin on disk testing of the present invention;

FIG. 15 is a post-test optical microscope image of pin on disk testing of the present invention;

FIG. 16 is a post-test optical microscope image of pin on disk testing of the present invention;

FIG. 17 is a graph of height verses distance as indicated in FIG. 15 of pin on disk testing of the present invention;

FIG. 18 is a graph of height verses distance as indicated in FIG. 16 of pin on disk testing of the present invention;

FIGS. 19(a) and (b) are graphs showing cyclic loading fretting corrosion test results for a CoCr-CoCr modular head-neck couple subjected to increasing cyclic loads from 100 N to 3200 N;

FIG. 20 is a graph of typical fretting current response for a similar head-neck taper interface, where the test is identical to the tests reported above, except the test was performed on a couple that had been previously subjected to one million cycles of fretting corrosion loading and then retested for its onset load;

FIG. 21 is a graph of current over time during in vitro testing of an embodiment of the present invention;

FIG. 22 is a graph of current over time during in vitro testing of an embodiment of the present invention;

FIG. 23 is a graph of current over time during in vitro testing of an embodiment of the present invention;

FIG. 24 is a graph of average current verses nominal maximum load during in vitro testing of an embodiment of the present invention;

FIG. 25 is a series of SEM images of a laterally positioned self-reinforced composition after in vitro testing of an embodiment of the present invention;

FIG. 25 is a series of SEM images of a medially positioned self-reinforced composition after in vitro testing of an embodiment of the present invention;

FIG. 27 is a digital optical microscopy image taken after in vitro testing of an embodiment of the present invention;

FIG. 28 is a digital optical microscopy image taken after in vitro testing of an embodiment of the present invention;

FIG. 29 is a graph of height verses distance measured as indicated in FIG. 27 after in vitro testing of an embodiment of the present invention.

FIG. 30 is a graph of height verses distance measured as indicated in FIG. 28 after in vitro testing of an embodiment of the present invention.

### DETAILED DESCRIPTION OF THE INVENTION

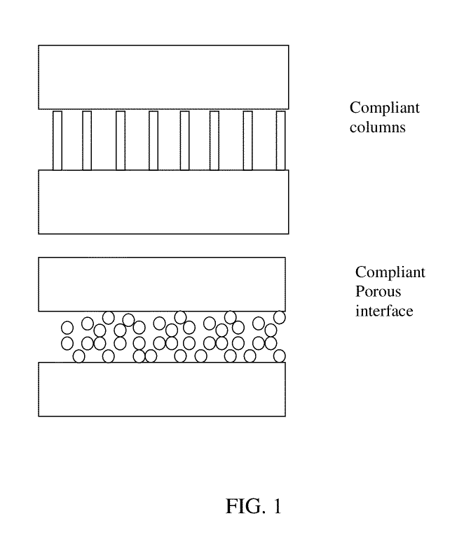

Referring now to the drawings, wherein like reference numerals refer to like parts throughout, there is seen in FIG. 1 a schematic of surface geometric structures to provide high interfacial adhesion and put the interface into the sticking regime of fretting, while allowing good exchange of fluid with the outside regions and preserving the overall mechanical integrity of the surface. As seen in FIG. 1, the present invention includes micro-columnar and/or porous surface structures for fretting interfaces. These surfaces provide interfacial compliance that is high enough so that relatively low normal forces between attached columns will result in high sticking friction and thus limit fretting motion between interfaces. Additionally, the space developed between columns will provide a free exchange of fluid from inside the crevice to the outside, reducing or eliminating the conditions under which the crevice solution changes can propagate crevice corrosion behavior.

Other approaches for use with the present invention may include micropatterning the surface and patterned dissolution, selective laser sintering of fine particles to the surface, or other techniques. For example, electrochemical surface treatment approaches may be used to first pattern the surface with a mask to insulate regions, while adjacent regions are subject to acid etch, electrochemical polish, or other approaches, to develop the appropriately compliant surface. These compliant micro-topographic surfaces (CMT-surfaces) may then be subjected to fretting corrosion experiments to determine effectiveness. Column geometry and density will be important considerations so some testing will be needed to maximize beneficial effects. Major variables may include column height to diameter, inter-column distance, and the anticipated fretting displacements.

Alternative surface chemistries that seek to reduce or eliminate fretting corrosion reactions are also encompassed by the present invention. These chemistries include the use of high performance polymer coatings, including the use of poly(ether ether ketone), referred to as PEEK, which is currently in use for spinal implant materials. Unreinforced polymer films are not likely suitable for the present invention because the polymers will not have adequate strength to withstand the high cyclic stresses over millions of cycles. However, highly oriented polymer fibers, such as those made from either ultra-high molecular weight polyethylene, UHMWPE or PEEK will be able to better withstand the loading and motion environment. Using a thin film of polymer will allow a direct application to current modular taper designs and indeed could be even used to retrofit current tapers at revision surgery.

High temperature, melt-spinning technologies for PEEK resins may be used to place thin films of PEEK onto roughened metal surfaces to create 20 μm thick interlayers to inhibit direct contact between metal surfaces and to eliminate the corrosion reactions in the crevice. Alternative approaches include hard ceramic layers. However, because of the difficult geometry, line-of-sight techniques like plasma spraying are not suitable. High temperature methods, like oxygen diffusion hardening, may thus be suitable.

The present invention also includes the use of UHMWPE fibers which are in the 20 to 40 μm thick range to interpose between the two hard surfaces of the taper interface. These fibers (or PEEK fibers) have very high tensile strength (approaching 1 to 2 GPA) and can be arrayed in the taper to transfer load across the interface while also providing fluid exchange paths to reduce solution chemistry changes. In this approach of the present invention, high strength polymer fibers which have the properties of high fracture resistance, insulating character, low hardness, high tensile strength, chemically inert can be interposed either in a parallel fashion or as a weave between the two sides of the modular taper. These high strength fibers will not easily degrade chemically or mechanically and will keep the two parts from making direct contact and will allow for more facile exchange of solution within the crevice. All of these effects will act to reduce or eliminate the conditions where fretting crevice corrosion can arise or become so severe as to result in adverse biological effects.

The geometry of SRC films could also have small slots or openings that will provide for easy fluid exchange with the outside environment, thereby reducing the acidification of the crevice solution and lowering the overall attack of the alloy. Because these SRC films will be in highly constrained geometries, it will be difficult for them to creep through to make direct metal-metal (or metal-ceramic) contact. While certain SRCs are proposed, it should be understood that other suitable polymer-based films could be used, provided that the films satisfy the requirements of being high strength, thin films with high electrical resistance and low abrasion potential with the substrate metal surfaces.

### EXAMPLE 1

One embodiment of the present invention involves use a thin SRC film made from PEEK. The basic concept is to melt spin PEEK fibers under the proper thermal and flow conditions to result in a highly oriented polymer structure that will impart significantly improved mechanical properties to the polymer. Then, these fibers can be hot compacted into a self-reinforced composite film with a thickness in the range of 10 to 200 μm (ideally between 20 and 100 μm).

The fibers of PEEK were made by melt spinning PEEK, at a temperature range of 350 to 370° C. PEEK polymer in pellet form is placed into an extrusion chamber and heated to the melt temperature. Then, the polymer is pushed through a small spinerette hole and taken up on a rotating drum that draws the polymer fiber down to a fine diameter (20 to 100 μm is typical). These fibers of PEEK can then be drawn further after spinning to further increase the strength and molecular orientation of the polymer chains in the fiber. Post-drawing strains between 5 and 60 percent are possible depending on the polymer, fiber and spinning conditions used.

Once sufficient fiber has been generated, self-reinforced composite PEEK (SRC-PEEK), or SRC-PE (SRC made from UHMWPE) can be fabricated by taking the fiber and laying it out in a metal mold in a unidirectional continuous fiber fashion and then placing a second metal plate over top. The thickness of the film will be governed by the diameter of the fibers used and the number of fibers in the film thickness. Optimally, a thin, continuous fiber reinforce film will require at least one fiber diameter, and up to two or three, to make a fully consolidated film. The two metal platens (metal plates) are then heated to a temperature near to or above the melt temperature of the PEEK and pressed together (or pressed, then heated). This will result in high temperature diffusional interactions between the adjacent fibers that will drive a bonding or fusion interaction to cause the fibers to incorporate into a film-based SRC-PEEK material but will retain much of the properties and molecular orientation of the fibers.

### EXAMPLE 2

Self-reinforced composite ultrahigh molecular weight polyethylene (SRC-PE) is a composite that consists of very high strength fibers of UHMWPE that are consolidated either directly without an interposing matrix, or with the addition of lower molecular weight PE powder that serves of the matrix. Direct SRC-PE is made in a similar fashion to the SRC-PEEK described above, however, the temperatures used are lower since the melt temperature is lower. High strength UHMWPE fibers, known commercially as SPECTRA®, can have tensile strengths that are 50 to 100 times the strength of un-oriented UHMWPE and can reach into the GPa range. SRC-PE films can be made as described above for SRC-PEEK films. Fibers of UMWPE are aligned in a mold and hot pressed at or near the melt temperature of the fiber. This drives a diffusion bonding interaction between fibers without complete loss of the molecular orientation within the fiber itself. These films can then be used as the interposing polymer layer between modular taper components.

### EXAMPLE 3

In this example, two metallic surfaces (both made from one of several medical alloys, e.g., 316L SS, Ti, and CoCr) are mounted in a fretting corrosion test system as set forth in Swaminathan, V, Gilbert, JL, “Fretting Corrosion of CoCrMo and Ti6A14V Interfaces,” Biomaterials 33 (2012), pp. 5487-5503, hereby incorporated by reference in its entirety. In testing such as this, a metallic pin is brought into contact under controlled load to an alloy disk. With the two immersed in a physiologically representative solution, the corrosion response of the couple can be monitored using voltage control current monitoring tests (using an electrochemical test system known as a potentiostat). When fretting abrasion of oxide films occurs (small scale cyclic relative motion), the repassivation reaction generates currents that can be detected and measured that directly reflect the extent and severity of the corrosion reactions. The pin is then cyclically slid a controlled distance (typically in the 50 um range) under load and the fretting corrosion currents are measured. If no currents are generated, then there is no mechanically assisted corrosion taking place and the surfaces are not having their oxide films abraded and repassivated. This is the optimal outcome for these interfaces and the overall objective of any solution to modular taper fretting crevice corrosion should demonstrate this lack of corrosion response to the mechanical-electrochemical interacation.

In this example, varying numbers of individual PEEK fibers (between 1 and 10) were interposed between the two metal components and fretting corrosion testing was performed with varying loads between 0.5 N and 30 N (nominal contact stresses between 5 MPa and 300 MPa) were applied. The pin was then cyclically slid back and forth while the metal parts were held at a fixed potential and the corrosion current response was monitored. Then, the load applied was increased and the test repeated up until 30 N was reached. After testing, the data was analyzed to see if and how much the corrosion currents increased as the load increased. The results were compared to tests of the same alloy combination but without any fibers placed between the pin and the disk.

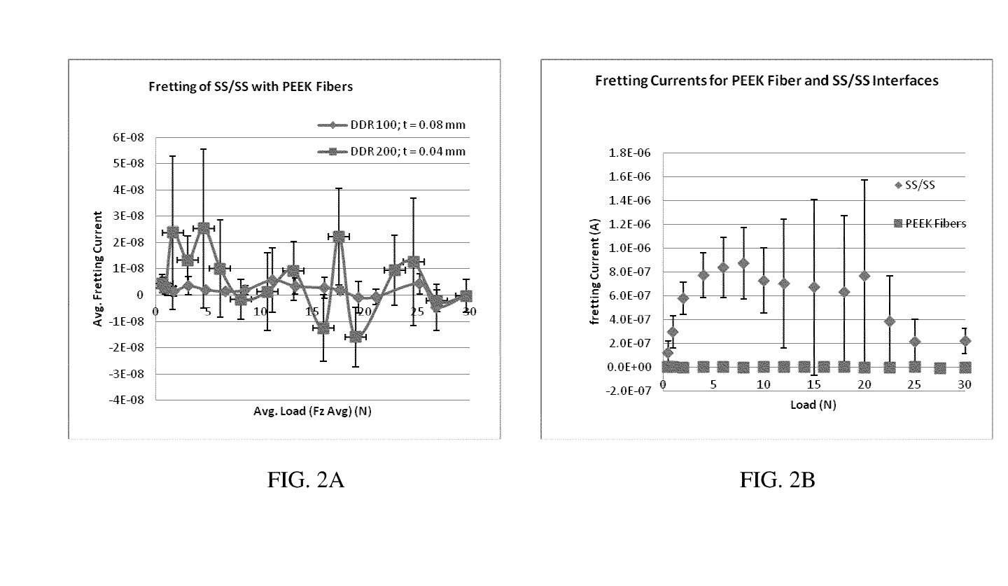

The results of pin-on-disk fretting corrosion testing are shown in FIG. 2. The fretting corrosion reaction across the entire range of loads explored showed no increase in currents compared to the non-fretted condition. This is the case for the two fiber types tested. When compared to stainless steel interfaces, see FIG. 2(b), one can see the complete lack of response of the PEEK fiber interposed between metals compared to the SS/SS interfaces which showed fretting currents rising about 100 times compared to the SRC-PEEK test results.

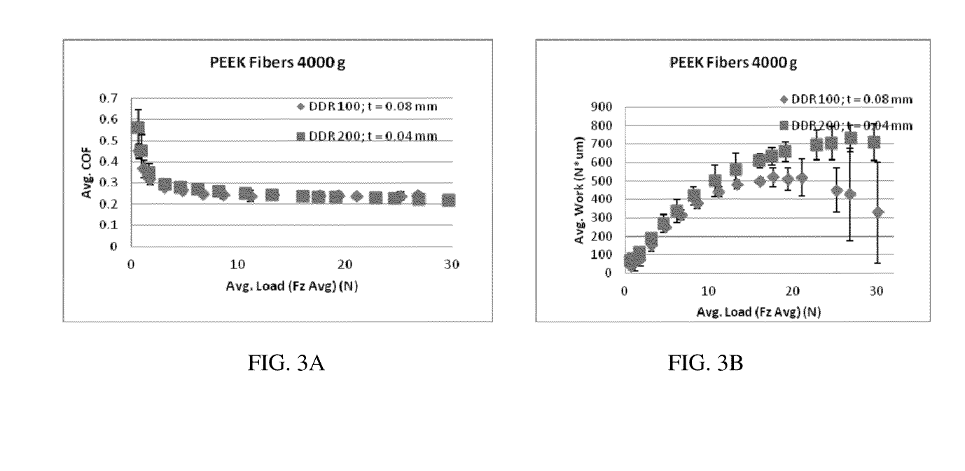

The frictional interaction and the work per cycle of fretting are shown in FIGS. 3(a) and 3(b), respectively. For both fiber types tested, the friction is consistent and the work per cycle results, see FIG. 3(b), indicate that the pin continued to slide relative to the disk throughout the entire loading range. It should be noted that these loads represent nominal contact stresses on the order of 5 MPa (at the lowest loads) to over 300 MPa at the higher end. This implies that these fibers can remain intact under extremely high contact stresses and are able to prevent direct metal-to-metal contact and subsequent fretting corrosion.

FIG. 4 shows a scanning electron microscopic image of two PEEK fibers after fretting corrosion testing with a stainless steel disk. It can be seen that the fibers have been flattened out from the testing. The estimated area of fibers engaged, based on this image and the maximum load tested, indicates a nominal contact stress in the range of 300 MPa (30 N and 2 fibers at 50 um×1 mm).

These examples demonstrate that when a high performance polymer fiber based construct that is only a single fiber thick, or a few fibers thick, is placed between hard surfaces (one of which is metallic), the fretting corrosion interactions that typically arise and lead to severe modular taper corrosion reactions are significantly and dramatically suppressed. While this was demonstrated with PEEK fibers, it is understood that a range of high performance polymeric fibers may be used in this application including UHMWPE fibers, Kevlar, and others that have the combined attributes of high strength and toughness, and high electrical resistance.

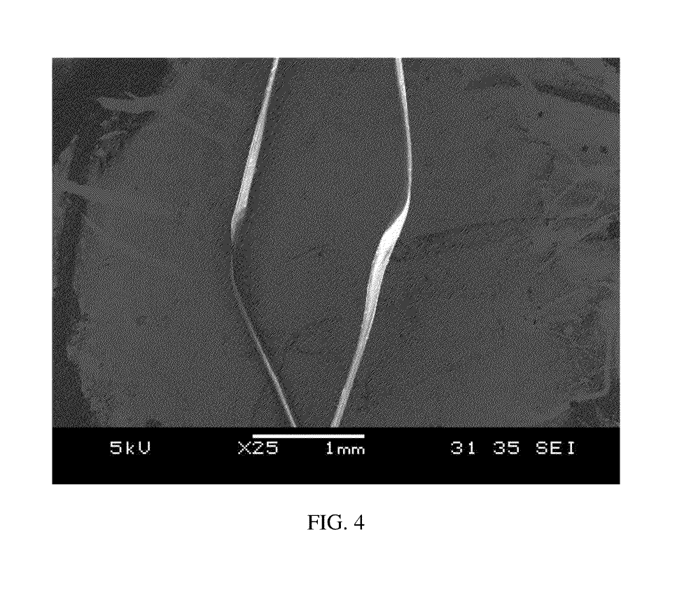

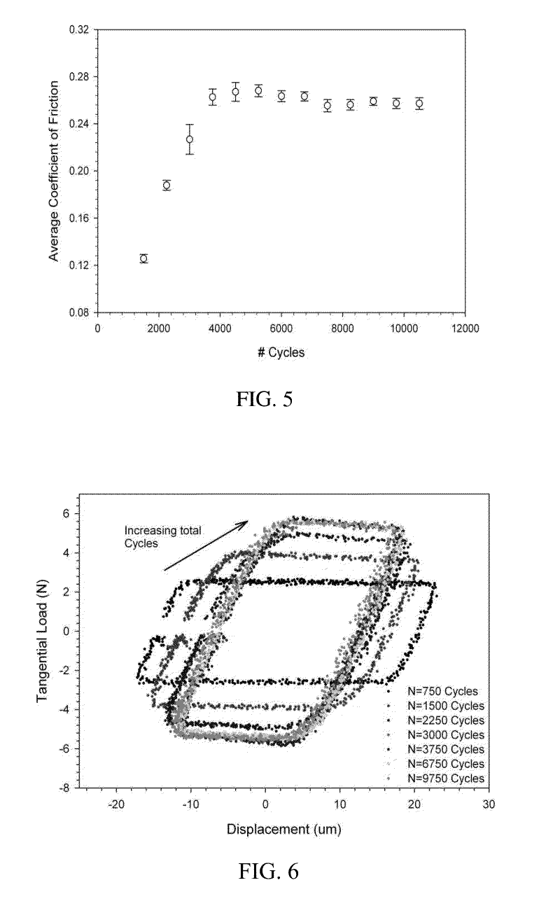

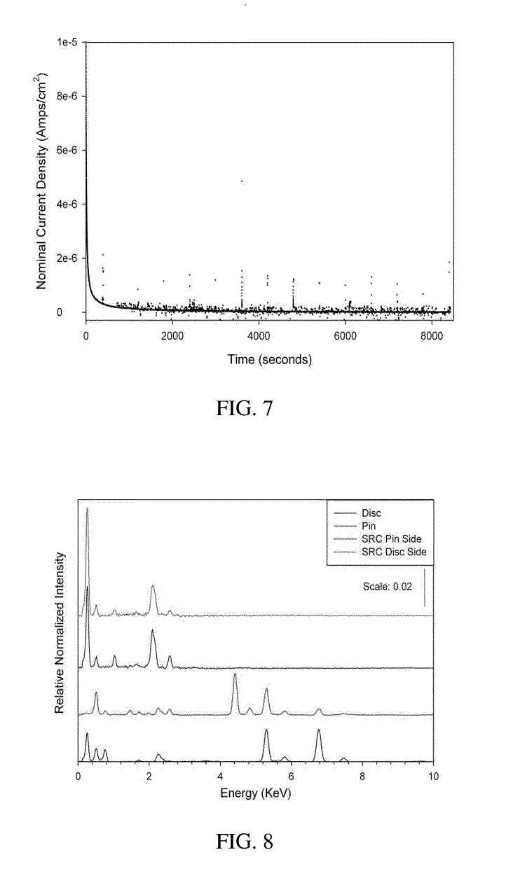

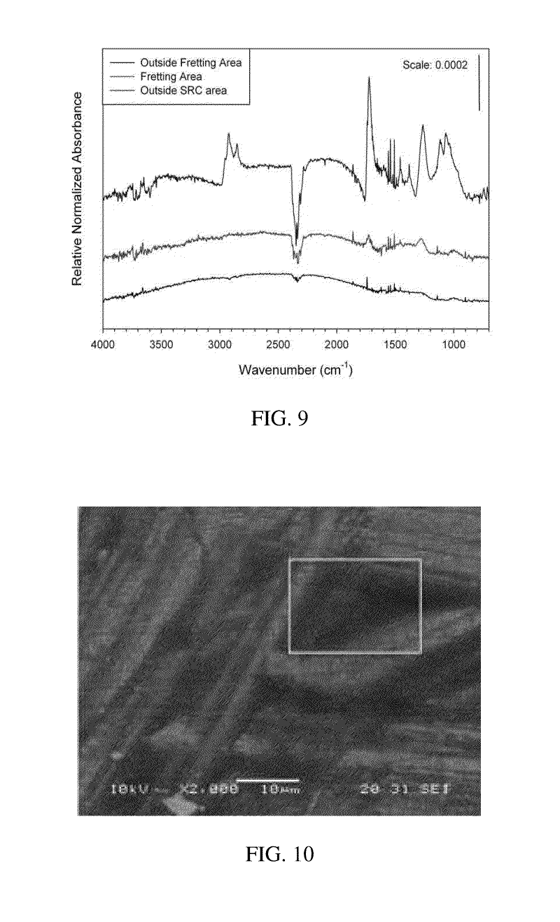

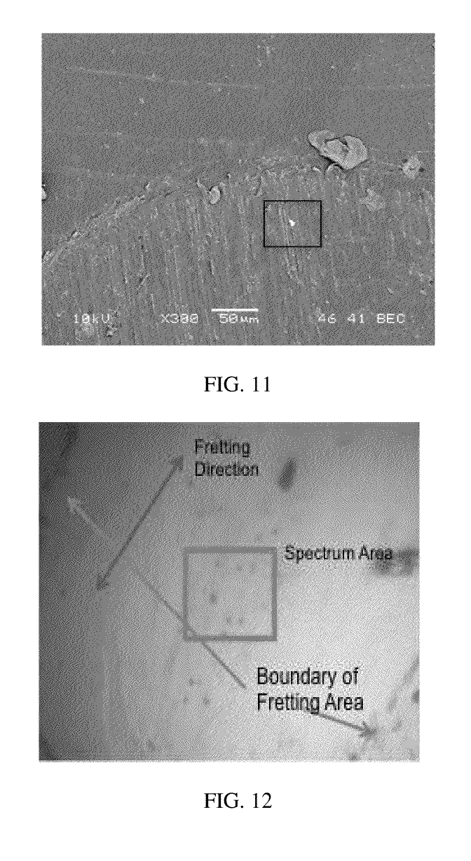

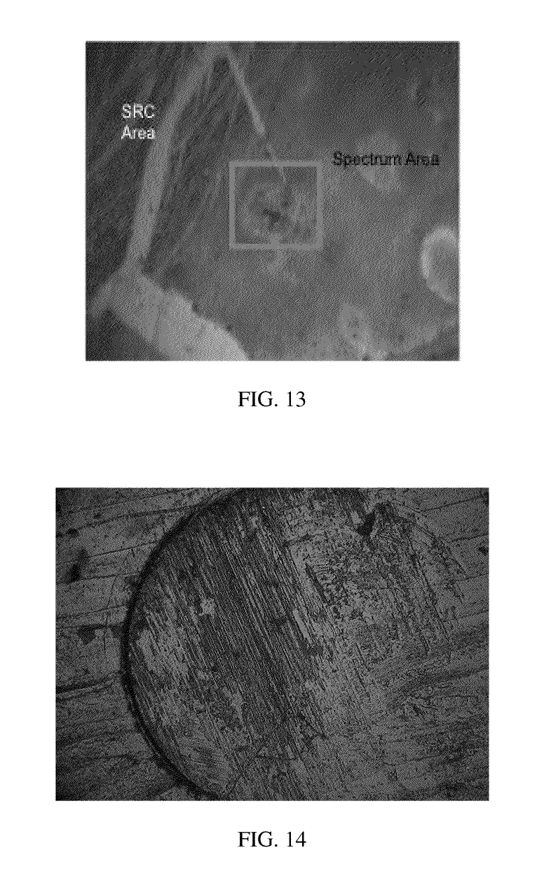

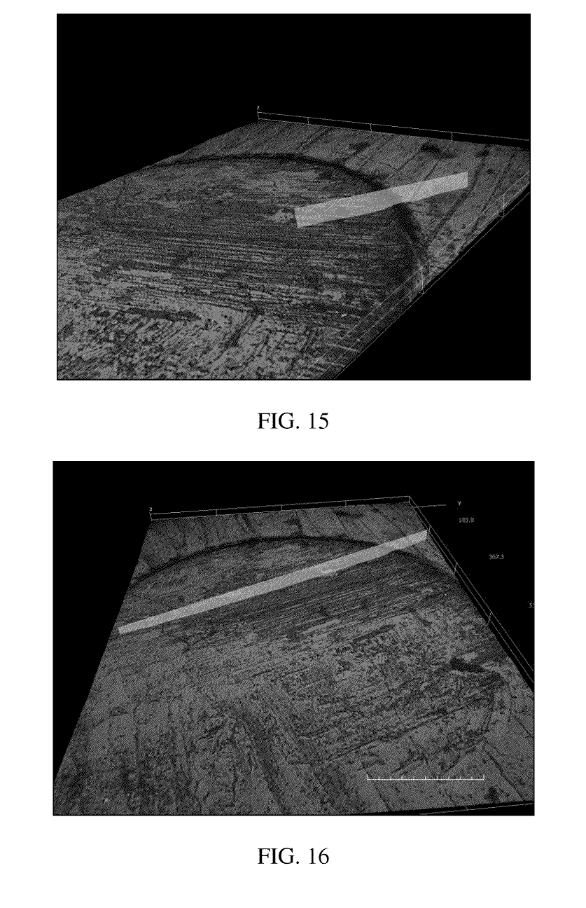

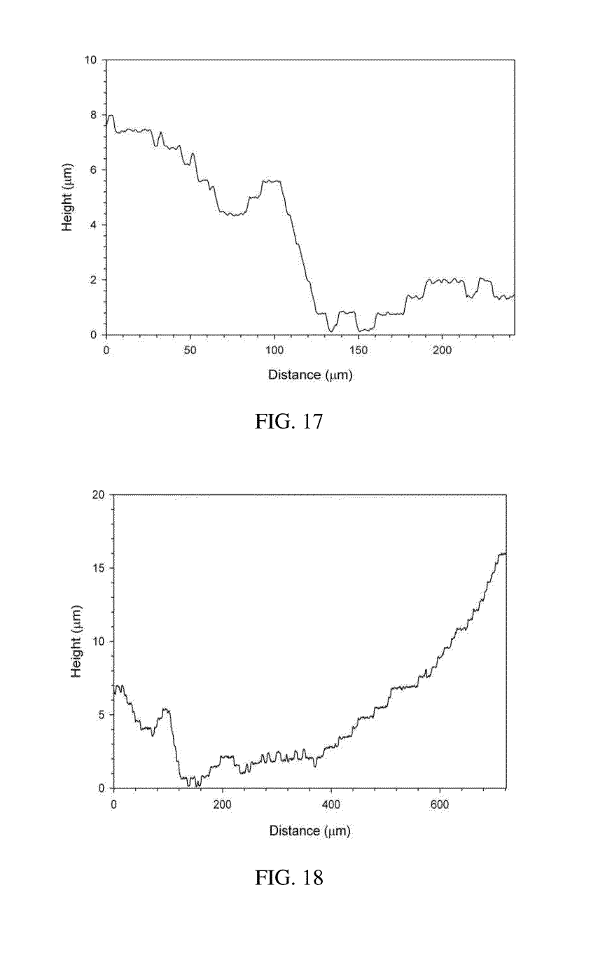

Additional pin on disk testing is seen in FIGS. 5 through 18. Testing was performed using SRC-PEEK fitted between a CoCrMo minor finish disk, and a Ti6A14V pin wet sanded. An electrochemical cell was filled with PBS and loaded to nominal contact stress of 100 MPa based on pin area. Fretting corrosion was tested by using over 10,500 cycles at 50 μm displacement and frequency 1.25 Hz, current evolution throughout the test is constantly monitored. Mechanical data (all loads, displacements, moments, coefficient of friction) collected every 10 minutes for 1 second at 600 Hz collection rate. The electrochemical results showed current density near zero for entire test, which suggests good integrity of the invention for the testing time scale. The mechanical results showed that displacement in “fretting loop” decreases over the test, as seen in FIG. 6, which suggests a change in surface morphology during test. As seen in FIG. 5, the coefficient of friction (COF) increased to a point early on, which mimics results seen in fretting loops. As seen in FIGS. 8 and 9, FTIR spectra of disk from three areas show very low signal and relative absorbance, indicating no presence of PEEK on alloy surface. Referring to FIGS. 10 though 13, SEM images show no evidence of alloy or oxide on surfaces. As seen in FIGS. 14 though 16, optical microscopy showed a clear imprint of pin on SRC-PEEK surface and topographical changes were easily captured with line profiling tool, with the results shown in FIGS. 17 and 18, and supports the above finding of increased COF and decreased tangential displacement. As in in vitro tests discussed below, damage appears as plastic deformation rather than wear, which was further supported by chemical surface analyses.

### EXAMPLE 4

In this example, SRC-PEEK films, fabricated as described above, of approximately 100 μm thickness were placed between the head (CoCr alloy) and neck (316L SS) of a total hip prosthesis construct. These SRC-PEEK films were cut into three separate strips because they were fabricated as flat strips, and were placed equidistant to each other around the circumference of the taper with the fibers oriented along the cone axis. Because the primary direction of fretting motion is also along the cone axis, the fibers oriented this way will maximally resist the fretting motion and stresses. The head was then hammered down onto the neck to secure the construct.

Then, the construct was subjected to cyclic mechanical testing while immersed in phosphate buffered saline, and the corrosion reactions were monitored using potentiostatic methods. In these tests, the voltage of the implant construct was held fixed and the currents generated by electrochemical processes were monitored. If fretting corrosion reactions are present, the reactions would be detected as an increase in the current measured in the three-electrode potentiostatic test.

The prosthesis construct was mounted in an acrylic mount such that the head-neck taper is exposed and the head component is partially immersed in the PBS. The orientation of the device is meant to simulate the single-leg stance phase of gait and a vertical compressive load (3 Hz, R=0.1) is applied to the head. A series of cyclic loading tests were performed in a servohydraulic test system where a cyclic compressive load is started at a low level (e.g., 100 N) and is incremented every three minutes (540 cycles) in 100 N increments until 1000 N and then 200 N increments up to 3200 N. The currents were then monitored continuously throughout the testing. If/when fretting corrosion begins to occur, the currents will rise above their resting level and the load at onset can be determined. Additionally, the currents generated at 3200 N can be used to make comparative assessments of the severity of the corrosion behavior.

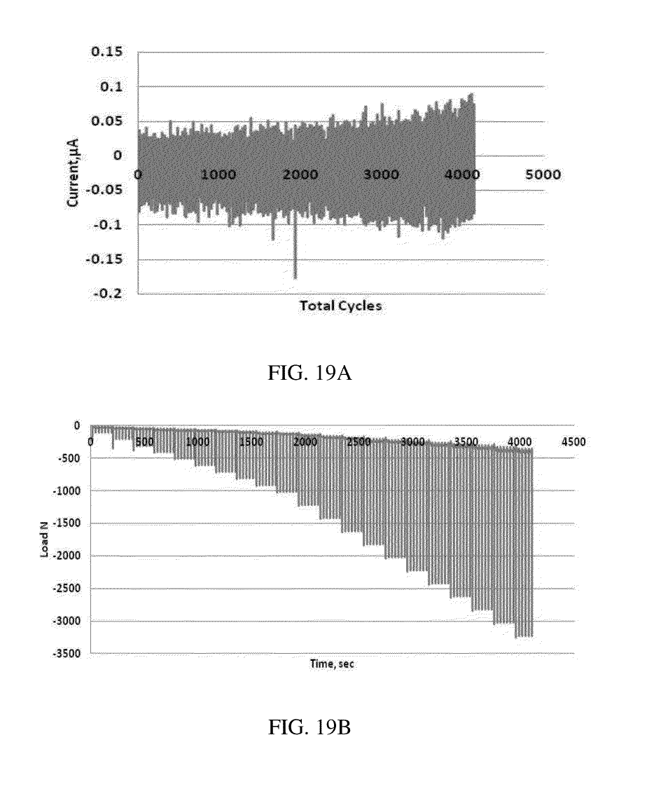

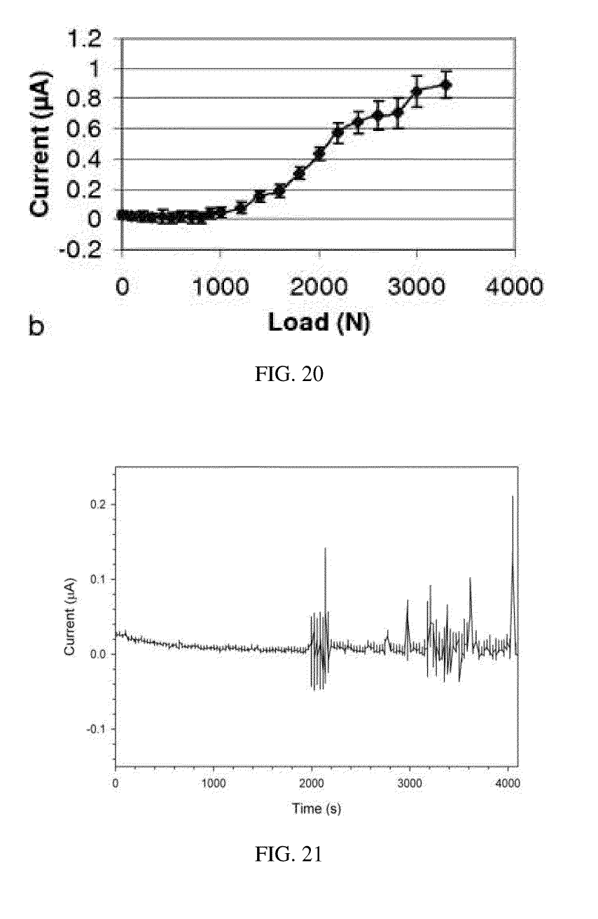

The results of these short term fatigue tests of modular head-neck taper junctions with SRC films are shown in FIG. 19A and 19B. The currents generated and the cyclic loads applied at each maximum load level are presented. The overall corrosion currents never demonstrated the onset of fretting corrosion with the SRC-PEEK films interposed between head and neck. These results show that fretting corrosion reactions are not taking place. This is in contrast to what is seen when direct metal-metal contact is made and similar loading is applied to the head-neck junction. A typical example of a metal-metal contact is shown in FIG. 20 for comparison to the SRC-PEEK film test of FIGS. 19A and 19B.

These results demonstrate that interposing a high performance self-reinforced fiber-based composite thin film according to the present invention comprises a means for suppressing or eliminating fretting crevice corrosion reactions at modular taper interfaces of total joint replacements. The advantages of this approach of the present invention are that one can retrofit existing tapers with an SRC film. The SRC film of the present invention can also be applied during revision surgeries where one side of the taper interface needs to remain in the patient. The SRC film can be placed over the taper and will provide a barrier between the new interface and the remaining surface.

While this example demonstrated the use of SRC-PEEK films, it is equally suitable to use other high performance fiber-based self-reinforced composites (or, indeed polymer-based composite films of a variety of combinations) to make this high strength, insulating film layer that can be interposed between the two metal surfaces (or metal and adjacent hard, ceramic surface.).

### EXAMPLE 5

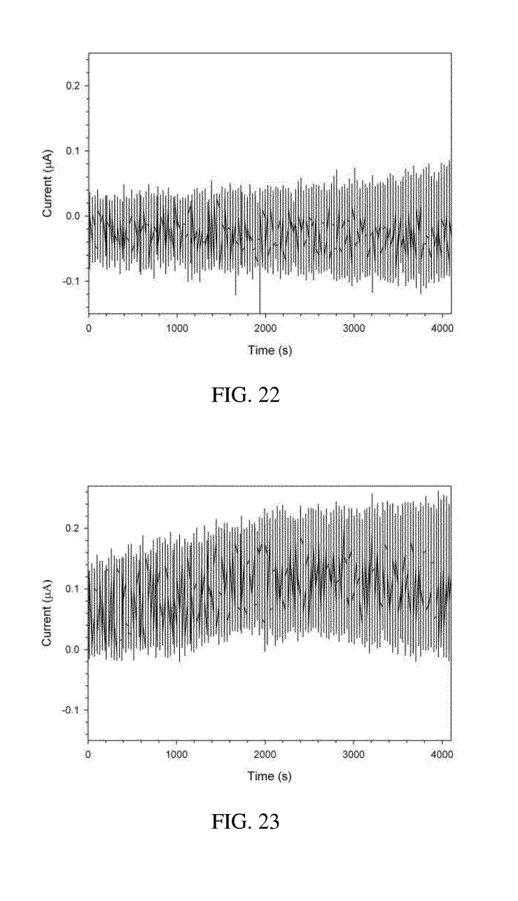

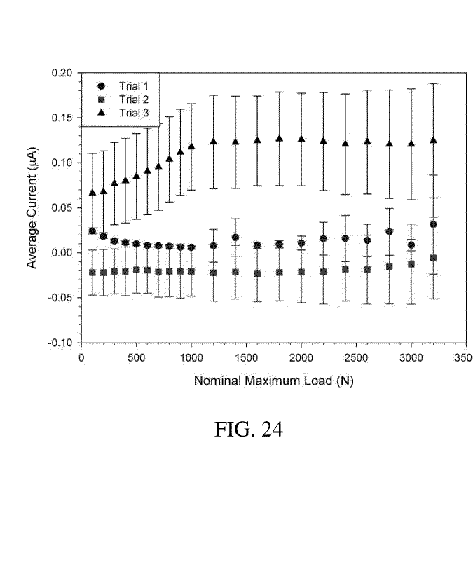

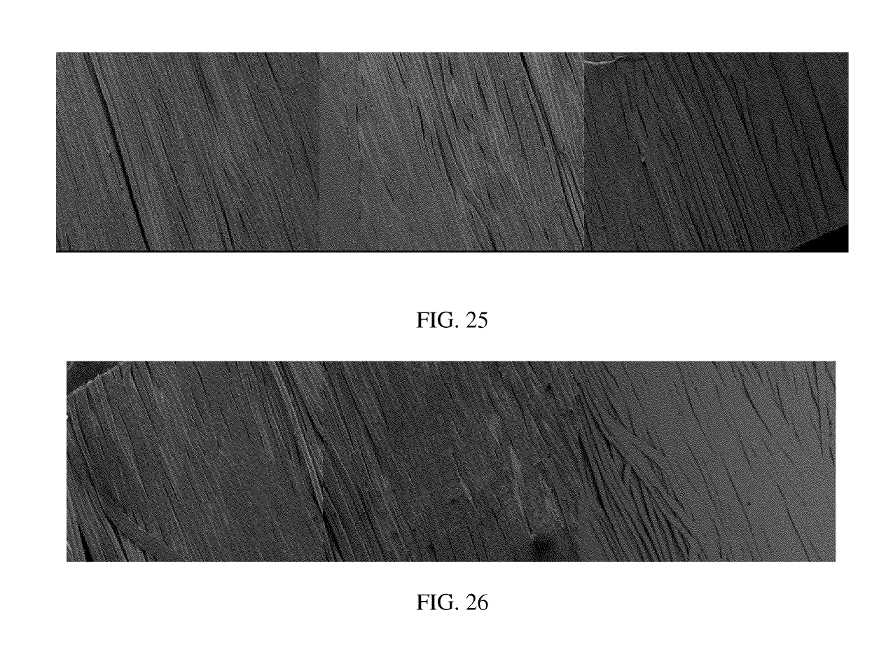

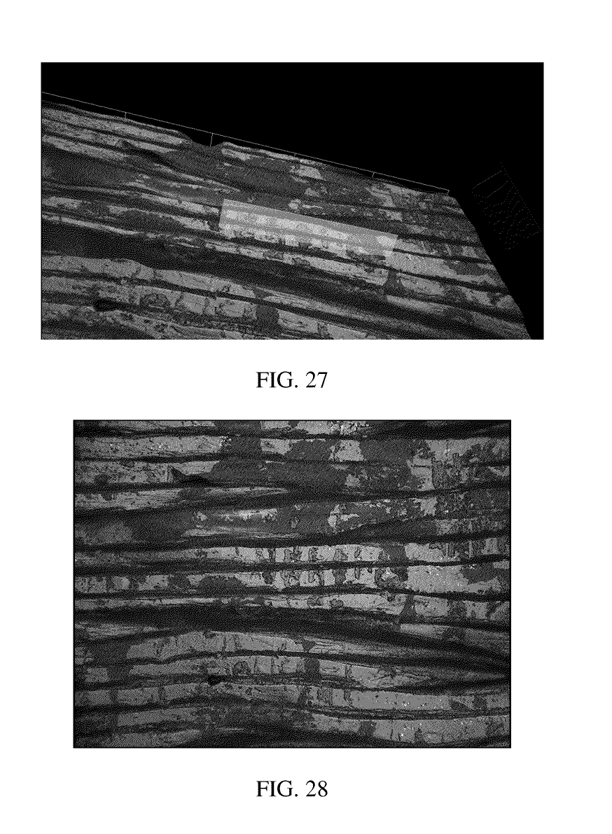

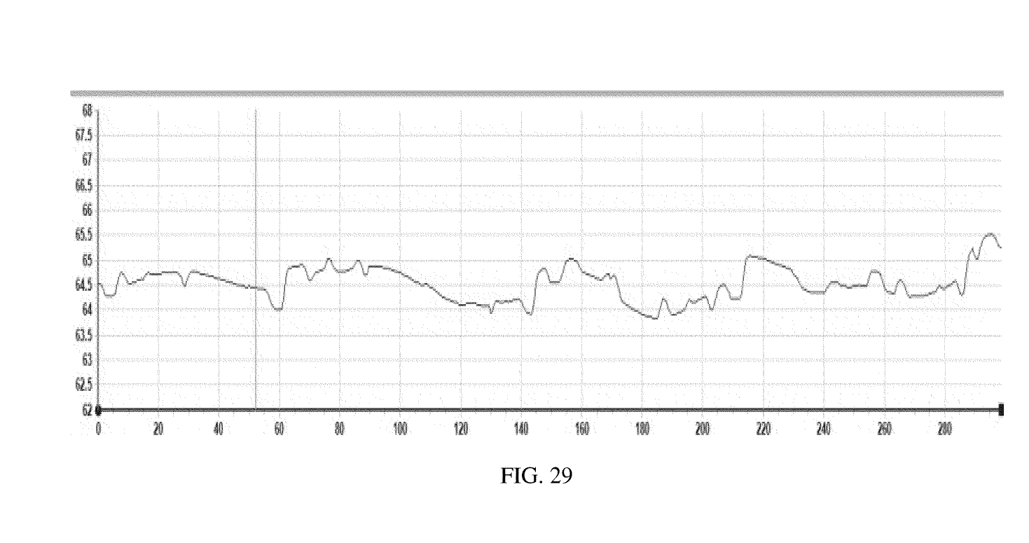

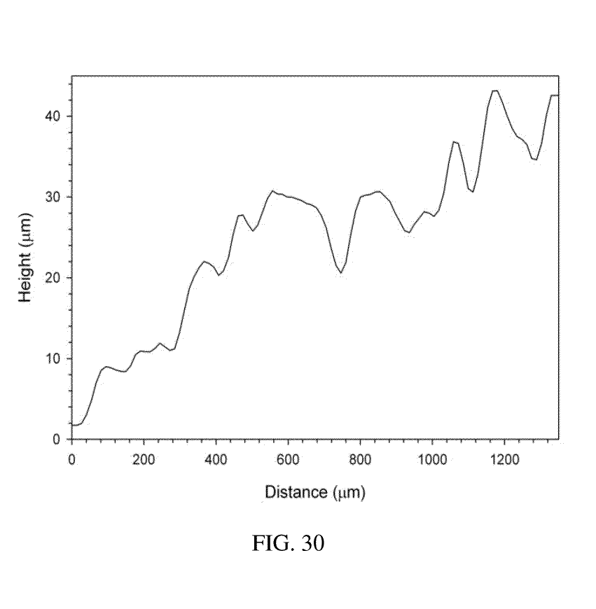

In other test of the present invention, a SRC-PEEK gasket was formed around a modular taper, potted in acrylic, and loaded into electrochemical cell. The femoral head/taper was established as a working electrode along with a carbon counter and an Ag/AgCl reference in phosphate buffered saline (PBS). Cyclic loading was performed from 100-1000 N in 100 N increments, and 1200-3200 N in 200 N increments (R=0.1). As seen in FIGS. 21 through 23, current was collected over time and plotted against the nominal maximum load. Referring to FIG. 24, overall current never climbed above ˜0.2 μA, indicating good electrical insulation between head and taper. SEM/EDS of SRC-PEEK, as seen in FIGS. 25 and 26, and digital optical microscopy, as seen in FIGS. 27 and 28, were used for quantification of damage post-trial. As seen in FIG. 25, the images of lateral SRC displayed a visible imprint of femoral head machine marks; however, damage appeared to be limited to some plastic deformation with no visible material removal. As seen in FIG. 26, the images of medial SRC showed damage that was slightly more severe in places but still, in large part, limited to the plastic deformation of fibers. Backscattered image analysis provided no indication of any alloy or oxide particles on the surface. As seen in FIGS. 27 and 28, and the corresponding measurement seen in FIGS. 29 and 30 taken along the areas indicated in FIGS. 27 and 28, respectively, digital optical microscopy revealed little to no relative motion between surfaces and thus tends to confirm that the present invention provides compliant sticking of the junction surfaces.

What is claimed is:

1. A coating for reducing fretting corrosion in an orthopedic implant, comprising at least one layer of a self-reinforced composite polymer.

2. The coating of claim 1, wherein said at least one layer of a self-reinforced composite polymer comprises a unidirectional continuous fiber of said polymer.

3. The coating of claim 2, wherein said at least one layer has an overall thickness of between 10 and 200 μm.

4. The coating of claim 3, wherein said self-reinforced composite polymer comprises poly ether-ether ketone.

5. The coating of claim 3, wherein said self-reinforced composite polymer comprises ultra-high molecular weight polyethylene.

6. A method of reducing fretting corrosion, comprising the steps of:
providing an orthopedic implant having a junction between first and second components; and
applying a coating comprising at least one layer of a self-reinforced composite polymer to at least one of said components.

7. The coating of claim 6, wherein said at least one layer of a self-reinforced composite polymer comprises a unidirectional continuous fiber of said polymer.

8. The coating of claim 7, wherein said at least one layer has an overall thickness of between 10 and 200 μm.

9. The coating of claim 8, wherein said self-reinforced composite polymer comprises poly ether-ether ketone.

10. The coating of claim 8, wherein said self-reinforced composite polymer comprises ultra-high molecular weight polyethylene.

11. A method of manufacturing an orthopedic implant having reduced fretting corrosion, comprising the steps of:
melt spinning a polymer to produce a polymer fiber having a predetermined diameter;
arranging the fiber in a mold in a unidirectional fashion to provide a fiber film having a predetermined thickness;
hot pressing the fiber film in the mold to fuse the fibers into a self-reinforced polymer film; and
affixing the self-reinforced polymer film to at least one component of a junction in a modular orthopedic implant.

12. The method of claim 11, wherein said predetermined diameter and said predetermined thickness of said fiber film are selected so that said resulting self-reinforced polymer film has an overall thickness of between 10 and 200 μm.

13. The coating of claim 12, wherein said polymer comprises poly ether-ether ketone.

14. The coating of claim 12, wherein said polymer comprises ultra-high molecular weight polyethylene.
